(19) United States
(12) Patent Application Publication
(10) Pub. No.: US 2021/0290160 A1
(43) Pub. Date: Sep. 23, 2021

(54) ON-SITE MONITORING DEVICE OF A HYDROCLEANER AND OF AN OPERATOR OF THE HYDROCLEANER, AND GLOBAL MONITORING SYSTEM

(71) Applicant: ORTEC EXPANSION, Aix-en-Provence Cedex (FR)

(72) Inventor: Daniel RIVARD, Paris (FR)

(21) Appl. No.: 17/206,805

(22) Filed: Mar. 19, 2021

(30) Foreign Application Priority Data
Mar. 20, 2020 (FR) 2002764

(51) Int. Cl. A61B 5/00; A61B 5/0205; B08B 3/02; B08B 13/00

(52) U.S. Cl. CPC A61B 5/6804; B08B 13/00; B08B 3/026; A61B 5/02055

(57) ABSTRACT

The on-site monitoring device may include a monitoring unit having at least one piece of clothing intended to be worn by the operator, who may be provided with sensors to measure values of parameters linked to the operator and/or to their environment and a transmission connection to communicate with a local monitoring unit mounted on the hydrocleaner. The local monitoring unit may be able to communicate with a control unit of a control station located at a distance from the worksite in such a way as to make it possible to remotely monitor the operator and the hydrocleaner.

### CROSS-REFERENCE TO RELATED APPLICATIONS

This application claims priority to French Application No. 2002764, filed Mar. 20, 2020, which application is incorporated by reference herein in its entirety.

### TECHNICAL FIELD

The present invention relates to a monitoring unit for an operator of a hydrocleaner, an on-site monitoring device comprising a hydrocleaner and said monitoring unit, as well as a global monitoring system (with remote intervention and control).

### PRIOR ART

It is known that a hydrocleaner of the industrial type is intended for industrial installation and equipment cleaning work. A hydrocleaner is in particular used during cleaning and pumping work on oil and oil-related sites.

It is known that a hydrocleaner used in oil and oil-related complexes has for object:
pumping oil products; and
performing various works (of stripping, of unclogging, refurbishing exchanger tubes, cleaning of enclosures or bins, . . . ) at high pressure.

These works can be dangerous for the operator.

In particular, in the case of pumping, there can be:
a physical risk, for example the sudden falling of the operator on the site, a dangerous atmosphere risk, a heart or respiratory risk; and
material risks due to abnormal operation of the hydrocleaner such as an abnormal rise in temperature of the motor of the carrier or of a pumping system of the hydrocleaner, an atmosphere in the environment of the hydrocleaner that is incompatible with operating safety, a loss of equipotentiality of a suction line.

Furthermore, in the case of high-pressure works, there are physical risks for the operator, due for example to the bursting of a hose or to the malfunction of a tool.

There is therefore a need for means that make it possible to monitor an operator of a hydrocleaner and/or equipment of the hydrocleaner in order to be able to detect a problem or a danger, or a risk of a problem or danger and thus be able to intervene from a remote station, on the hydrocleaner or come to the aid of the operator.

### DISCLOSURE OF THE INVENTION

The object of the present invention is to respond to this need. It first of all relates to a monitoring unit for an operator of a hydrocleaner.

According to the invention, said monitoring unit comprises:
at least one piece of clothing intended to be worn by the operator, said clothing being provided with a first set of sensors configured to measure values of parameters linked to said operator and/or to their environment; and
at least one radio transmitter configured to emit in the form of radio waves first information, and at least values measured by at least some of said sensors of said first set of sensors.

Thus, thanks to the sensors, the operator and their environment can be monitored (in real time), and thanks to the radio transmitter, the values measured can be transmitted to a control station for remote monitoring as stated hereinbelow.

Advantageously, said clothing corresponds to one of the following elements: a worksuit, an undergarment.

Moreover, advantageously, said first set of sensors comprises sensors able to measure at least some of the following parameters of the operator:
one or more pieces of heart data (heart rate, blood pressure, etc.);
one or more pieces of respiratory data;
their body temperature;
their pulse;
their position.

Furthermore, advantageously, said first set of sensors comprises sensors capable of measuring at least some of the following parameters of the environment of the operator:
the presence of at least one gas;
the temperature;
the wind.

The present invention also relates to an on-site monitoring device.

According to the invention, said on-site monitoring device comprises at least one monitoring unit such as the one described hereinabove and a local monitoring unit intended to be mounted on a hydrocleaner. This local monitoring unit comprises at least one radio receiver configured to be able to receive the first information emitted in the form of radio waves by the radio transmitter of said monitoring unit and a radio transmitter capable of emitting in the form of radio waves second information (intended for a remotely-located control station).

Advantageously, said local monitoring unit comprises a second set of sensors intended to be mounted on the hydrocleaner, said second set of sensors comprising sensors configured to measure values of parameters linked to said hydrocleaner and/or to the environment thereof.

Furthermore, advantageously, said second set of sensors comprises sensors capable of measuring at least some of the following parameters of the hydrocleaner:
parameters of a motor of the hydrocleaner;
parameters of a piece of equipment of the hydrocleaner.

In addition, advantageously, said second set of sensors comprises sensors capable of measuring at least some of the following parameters of the environment of the hydrocleaner:
the presence of at least one gas;
the temperature;
the wind.

Moreover, said on-site monitoring device comprises:
at least one visual and/or audible alert element; and/or
a central unit for processing the values measured; and/or
the hydrocleaner on which said local monitoring unit is mounted.

The present invention also relates to a global monitoring system, allowing for remote intervention and control.

According to the invention, said global monitoring system comprises at least one on-site monitoring device such as described hereinabove, and a remote control unit. This remote control unit comprises at least one second radio transmission unit able to at least receive second information emitted by a first radio transmission unit of said local monitoring unit.

Advantageously, the remote control unit comprises at least one of the following elements:
at least one visual and/or audible alert element;
at least one display element;
at least one data processing element.

Moreover, advantageously, the remote control unit comprises a control unit configured to generate control orders making it possible to remotely control at least one piece of equipment of the hydrocleaner, said control orders being emitted by said second radio transmission unit and received by said first radio transmission unit.

Furthermore, advantageously, the remote control unit comprises an alert unit configured to generate orders for triggering an alert making it possible to remotely control at least one alert element of the on-site monitoring device, said orders for triggering an alert being emitted by said second radio transmission unit and received by said first radio transmission unit.

### BRIEF DESCRIPTION OF THE FIGURES

The accompanying figures will indeed help to understand how the invention can be carried out. In these figures, identical references designate similar elements.

FIG. 1 is a block diagram of a particular embodiment of a global monitoring system.

FIG. 2 is a diagrammatical view of a particular example of a hydrocleaner and of an operator carrying out pumping.

FIG. 3 is a diagrammatical view of a suit of an operator, provided with sensors.

FIG. 4 is a diagrammatical view of an undergarment of an operator, provided with sensors.

FIG. 5 is a diagrammatical view showing an operator using a high-pressure gun.

FIG. 6 is a diagrammatical view of a remotely located control (and intervention) station.

### DETAILED DESCRIPTION

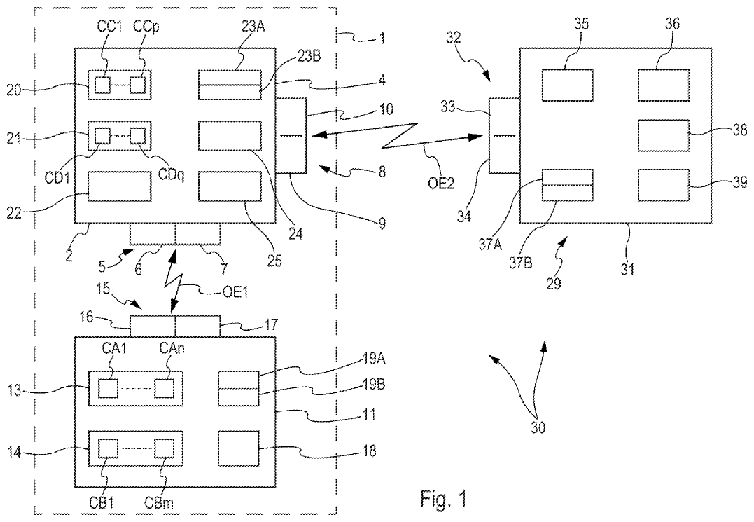

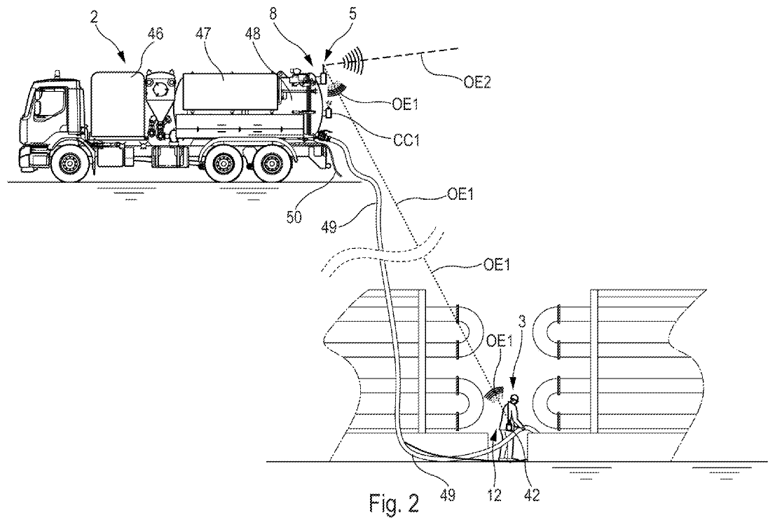

The device 1, showing the invention and diagrammatically shown in FIG. 1, is an on-site monitoring device, intended for monitoring an operation site of a hydrocleaner 2 (FIG. 2).

More precisely, the device 1 is intended for monitoring at least some of the following elements of the operation site: the hydrocleaner 2, the environment of the hydrocleaner 2, at least one operator 3 (FIG. 2) of the hydrocleaner 2 and the environment of the operator 3 (in particular if the operator 3 is far from the hydrocleaner 2).

Said device 1 (for on-site monitoring) comprises a local monitoring unit (hereinafter “unit 4”) that is mounted on the hydrocleaner 2.

The unit 4 (for local monitoring) comprises a radio transmission unit 5 comprising at least one radio receiver 6. The radio transmission unit 5 is intended for communication with the operator as stated hereinbelow. The radio receiver 6 is configured to be able to receive information emitted in the form of low-frequency radio waves OE1.

In a first embodiment, the radio transmission unit 5 comprises only the radio receiver 6.

In a second embodiment, the radio transmission unit 5 comprises, in addition to the radio receiver 6, a radio transmitter 7. The radio transmitter 7 is configured to be able to emit information emitted in the form of radio waves OE1.

Furthermore, the unit 4 (for local monitoring) comprises a radio transmission unit 8 comprising at least radio transmitter 9. The radio transmission unit 8 is intended for communication with a remote control station 29 as stated hereinbelow. The radio transmitter 9 is configured to be able to emit information in the form of radio waves OE2.

In a first embodiment, the radio transmission unit 8 comprises only the radio transmitter 9.

In a second embodiment, the radio transmission unit 8 comprises, in addition to the radio transmitter 9, a radio receiver 10. The radio receiver 10 is configured to be able to receive information emitted in the form of radio waves OE2.

In an alternative embodiment (not shown), the device 1 comprises, instead of both radio transmission units 5 and 8, only one radio transmission unit for communication, both with the worksite of the operator 3 and with the remote control station 29.

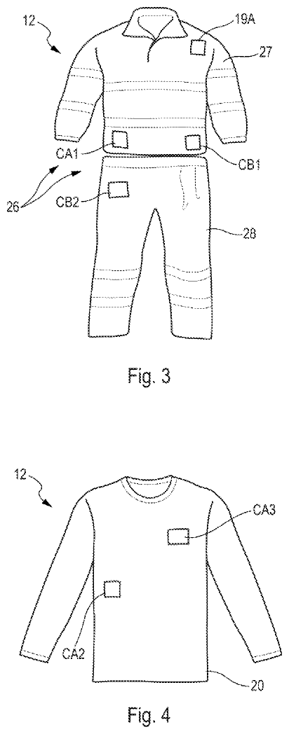

Moreover, the operator 3 is provided with a monitoring unit 11 (personal) that is integrated, preferably, at least partially in at least one piece of clothing 12 (FIGS. 3 and 4) worn by the operator 3.

The monitoring unit 11, which is intended for monitoring the operator and their working environment, is provided with a set 13 of sensors. This set 13 comprises, as shown in FIG. 1, a plurality of sensors CA1 to CAn, n being an integer. These sensors CA1 to CAn are configured to measure values of parameters linked directly to the person who is the operator.

More precisely, said set 13 can comprise, among the sensors CA1 to CAn:
one or more sensors to measure one or more pieces of heart data (heart rate, blood pressure, etc.) of the operator 3, in particular to be able to detect a heart accident of the operator or an immediate risk of heart accident;
one or more sensors to measure one or more pieces of respiratory data of the operator 3, in particular to be able to detect a respiratory insufficiency;
a thermal sensor, in particular a thermometer, for measuring the body temperature of the operator 3 and to be able to detect fever;
a sensor for measuring the pulse of the operator 3, for example in order to be able to detect an excessively high heart rate;
one or more sensors, for example gyrometers and/or accelerometer, for measuring the position of the operator, and optionally some of their movements. These sensors can, in particular, be configured to measure data that is representative of a falling of the operator 3.

The monitoring unit 11, which is intended for the monitoring of the operator and of their working environment, is also provided with a set 14 of sensors. This set 14 comprises, as shown in FIG. 1, a plurality of sensors CB1 to CBm, m being an integer. These sensors CB1 to CBm are configured to measure values of parameters linked to the working environment of the operator, in order to able to evaluate in particular the quality of the surrounding atmosphere.

More precisely, said set 13 can comprise, among the sensors CB1 to CBm:
one or more sensors to measure, in the working environment of the operator 3, the presence of one or more gases, potentially harmful; and/or
a thermal sensor, in particular a thermometer, to measure the temperature of the working environment of the operator 3 and to detect for example an abnormally high temperature; and/or
a sensor for measuring the wind, in the working environment of the operator 3, in particular in case of work outdoors.

The monitoring unit 11 also comprises a radio transmission unit 15 comprising at least radio transmitter 16. The radio transmission unit 15 is intended for communication with the unit 4 of the hydrocleaner 2, as stated hereinbelow. The radio transmitter 16 is configured to be able to emit information emitted in the form of radio waves OE1 that are received by the radio receiver 6 of the monitoring unit 4 of the hydrocleaner 2.

In a first simplified embodiment, the radio transmitter 16 sends, as information, directly the values measured by at least some of the sensors of sets 13 and 14.

These values can be communicated, continuously or periodically, in particular according to their importance in the monitoring.

In a second embodiment, the monitoring unit 11 comprises a processing element 18 that is connected to at least some sensors of sets 13 and 14 and that performs pre-processing of certain measured values, for example a comparison of a measured value with a threshold value in order to detect an anomaly. In this second embodiment, the radio transmitter 16 sends information to the hydrocleaner 2 only in case of detection of an anomaly by the processing element 18 in order to inform about this anomaly.

Furthermore, a third embodiment corresponds to the suite of the preceding first and second embodiments.

Moreover, in a first embodiment, the radio transmission unit 15 comprises only the radio transmitter 16.

Furthermore, in a second embodiment, the radio transmission unit 15 comprises, in addition to the radio transmitter 16, a radio receiver 17. The radio receiver 17 is configured to be able to receive information emitted in the form of radio waves OE1 by the radio transmitter 7 of the unit 4 of the hydrocleaner, as stated hereinbelow.

Means are provided to secure the transmission between the radio transmission unit 15 and the radio transmission unit 8.

The monitoring unit 11 comprises at least one alert element (or warning device) 19A, 19B. This can be a visual alert element 19A which is more preferably worn by the operator 3 and which is for example attached to the vest 27 of a worksuit 26 (FIG. 3). This visual alert element 19A is able to emit a visual alert signal such as a blinking light. This can also be an audible alert element 19B, such as a siren, which is more preferably worn by the operator and that is capable of emitting an audible alert element.

The alert element or elements 19A and 19B can be triggered in different ways according to the embodiment considered. In particular:
the alert element or elements 19A and 19B can be directly connected to at least some sensors of sets 13 and 14 and be triggered as soon as a particular value is measured by one of the sensors, in particular when a threshold value is exceeded. This can, in particular, be the case when a sensor detects a harmful gas; and/or
the alert element or elements 9A and 9B can be linked to the processing element 18 and be triggered as soon as an anomaly is detected by the processing element 18, for example when the heart rate of the operator exceeds a certain value; and/or
the alert element or elements 9A and 9B can be triggered by an order emitted via the radio transmitter 7 of the hydrocleaner 2 and received by the radio receiver 17. As stated hereinbelow, such an order can be generated by a remotely-located control station 29.

At least one portion of the monitoring unit 11 is integrated into one or more pieces of clothing 12 worn by the operator 3 during their work.

This clothing 12 can correspond to a worksuit 26, as shown in FIG. 3. For the purposes of illustration, in the example of FIG. 3, the worksuit 26 comprising a vest 27 and trousers 28, is provided with sensors CA1, CB1 and CB2 arranged at different locations of said worksuit 26.

This clothing 12 can also correspond to an undergarment 20, as shown in FIG. 4. For the purposes of illustration, in the example of FIG. 4, the undergarment 20 is provided with sensors CA2 and CA3 arranged at various locations of said undergarment 20.

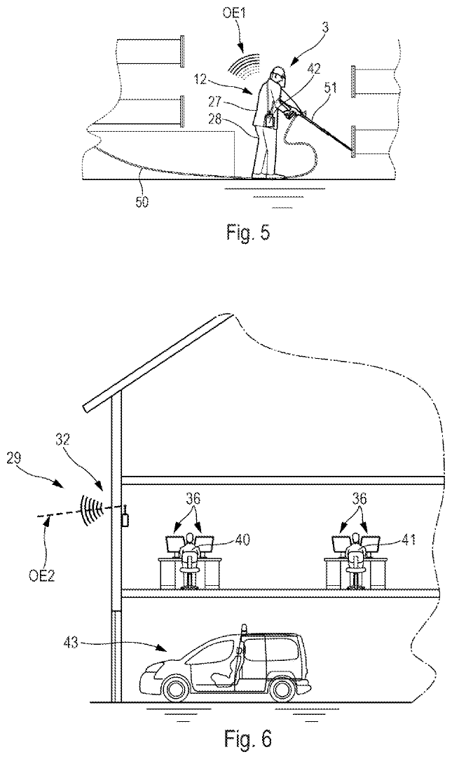

Some of the elements of the monitoring unit 11, for example an alert element, can also be integrated into a portable element 42 (FIGS. 2 and 5), such as a bag or another portable element, that the operator 3 carries or which is placed in the vicinity of the worker when they are working.

Moreover, the unit 4 (for local monitoring) which is also intended for monitoring the hydrocleaner 2 and the environment thereof, is provided with a set 20 of sensors. This set 20 comprises, as shown in FIG. 1, a plurality of sensors CC1 to CCp, p being an integer. These sensors CC1 to CCp are configured to measure values of parameters linked to said hydrocleaner 2. This in particular can be:
parameters of a motor of the hydrocleaner 2; and
parameters of a piece of equipment, for example of a pumping system, of the hydrocleaner 2.

More precisely, said set 20 can comprise, among the sensors CC1 to CCp, one or more sensors to measure one or more of the following parameters of the motor of the hydrocleaner 2:
an abnormal rise in the temperature of the motor; and/or
a loss of oil pressure of the motor; and/or
an abnormal speed of the motor.

Said set 20 can also comprise, among the sensors CC1 to CCp, one or more sensors to measure one or more of the following parameters of a piece of equipment of the hydrocleaner:
relating to a pumping system 46 (FIG. 2): an abnormal rise in the temperature of the motor of the pumping system 46, a loss of oil pressure for the drive of the pumping system 46, an abnormal speed of the pumping system 46; and/or
a failure of the filling level of a collection tank 48 (FIG. 2) of the hydrocleaner 3; and/or
a loss of the equipotentiality of a suction line.

The unit 4 is also provided with a set 21 of sensors. This set 21 comprises, as shown in FIG. 1, a plurality of sensors CD1 to CDq, q being an integer. These sensors CD1 to CDq are configured to measure values of parameters linked to the environment of the hydrocleaner 2.

More precisely, said set 21 can comprise, among the sensors CD1 to CDq:
one or more sensors to measure, in the environment of the hydrocleaner 2, the presence of one or more gases, potentially harmful; and/or
a thermal sensor, in particular a thermometer, to measure the temperature of the environment of the hydrocleaner 2 and to be able to detect an abnormally high temperature; and/or
a sensor for measuring the wind of the environment of the hydrocleaner 2.

Moreover the unit 4 also comprises a central unit 22 that is connected to at least some sensors of sets 20 and 21 and that performs processing of certain measured values, for example a comparison of a measured value with a threshold value in order to detect an anomaly.

The central unit 22 can also perform processing of values measured by sensors of sets 13 and 14 of the operator, values that are received by the radio receiver 7.

The unit 4 also comprises at least one alert element 23A, 23B. This can be a visual alert element 23A that is, preferably, mounted on the hydrocleaner 2 and that is capable of emitting a visual alert signal, such as a blinking light. This can also be an audible alert element 23B, such as a siren, which is preferably mounted on the hydrocleaner 2 and that is capable of emitting an audible alert element.

The alert element or elements 23A and 23B can be triggered in different ways according to the embodiment considered. In particular:
the alert element or elements 23A and 23B can be linked to at least some sensors of sets 20 and 21 and be triggered as soon as a particular value is measured by one of these sensors; and/or
the alert element or elements 23A and 23B can be linked to the central unit 22 and be triggered as soon as an anomaly is detected by the central unit 22; and/or
the alert element or elements 23A and 23B can be triggered on an order received by the radio receiver 10, from a remotely-located control station 29, as stated hereinbelow.

The device 1 (for on-site monitoring) is part of a remote control and optionally intervention system (hereinafter “system 30”), as shown in FIG. 1.

This system 30 (which is a global monitoring system) comprises, in addition to said device 1 (for on-site monitoring such as described hereinabove), a remote control unit 31 arranged at the remotely-located control station 29, shown in FIG. 6.

The remote control unit 31 is provided with a radio transmission unit 32.

The radio transmission unit 32 comprises, as shown in FIG. 1, at least radio receiver 33. The radio transmission unit 32 is intended for communication with the unit 4 as stated hereinbelow. The radio receiver 33 is configured to be able to receive information emitted in the form of radio waves OE2 by the radio transmitter 9 of the unit 4 (for local monitoring) of the hydrocleaner 3. Means are provided to secure the transmission between the radio transmission unit 32 and the radio transmission unit 8.

In a first embodiment, the radio transmission unit 32 comprises only the radio receiver 33.

In a second embodiment, the radio transmission unit 32 comprises, in addition to the radio receiver 33, a radio transmitter 34. The radio transmitter 34 is configured to be able to emit information (and in particular orders, commands or alerts) in the form of radio waves OE2 intended for the hydrocleaner 2, in particular to trigger an alert element or to take control of a piece of equipment of the hydrocleaner as stated hereinbelow.

The transmission between, on the one hand, the radio transmission unit 8 of the hydrocleaner 3 and, on the other hand, the radio transmission unit 32 of the control station 29, is secure.

The remote control unit 31 comprises a set of means that make it possible to inform and alert persons 40 and 41 (FIG. 6) installed (remotely from the worksite) at the control station 29 of an anomaly. These persons 30 and 31 are employed to remotely monitor both the operator, the hydrocleaner and the worksite, and to intervene remotely if necessary. Preferably, this is at least a technician 40 and a doctor 41 who are present at the control station 29 at least during the duration of the work of the operator 3.

The remote control unit 31 comprises a data processing element 35 configured to perform processing of certain measured values that are received by the radio receiver 34, for example a comparison of a measured value with a threshold value in order to detect an anomaly.

The remote control unit 31 also comprises at least one display element 36, such as a screen, that makes it possible to display measured unprocessed values and/or values processed by the central unit 22 or by the data processing element 35.

The remote control unit 31 also comprises at least one alert element 37A, 37B. This can be a visual alert element 37A that is installed in the control station 29 and that is capable of emitting a visual alert signal such as a blinking light. This can also be an audible alert element 37B, such as a siren, that is also installed in the control station 29 and that is capable of emitting an audible alert element.

The alert element or elements 37A and 37B can be triggered in different ways according to the embodiment considered. In particular:
the alert element or elements 37A and 37B can be triggered as soon as a particular value is measured by one of the sensors of sets 13, 14, 20 and 21; and/or
the alert element or elements 37A and 37B can be triggered as soon as an anomaly is detected by the central unit 22 or by the data processing element 35.

Moreover, in a particular embodiment, the remote control unit 31 comprises a control unit 38. This control unit 38 is configured to allow a person of the control station 29, in particular the technician 40, to generate control orders. These control orders are intended to remotely control at least one piece of equipment of the hydrocleaner, for example stop it is case of malfunction or danger.

The control orders are emitted by the radio transmission unit 32 of the control station 29 and received by the radio transmission unit 8 of the device 1 which transmits them to a control element 24 of the hydrocleaner 3 or to a control element 25 of a piece of equipment of the hydrocleaner 3.

Furthermore, in a particular embodiment, the remote control unit 31 comprises an alert unit 39. This alert unit 39 is configured to allow a person of the control station 29, in particular the technician 40, to generate orders for triggering an alert making it possible to remote trigger at least one alert element 19A, 19B, 23A, 23B of the device 1 (for on-site monitoring).

These orders for triggering an alert are emitted by the radio transmission unit 32 of the control station 29 and received by the radio transmission unit 8 of the device 1 which transmits them to the alert element concerned.

The persons who remotely monitor the worksite, in particular the technician 40 (for technical support and monitoring) and the doctor 41 (for medical support and monitoring), are therefore informed in real time of any anomaly, danger or accident occurring on the worksite and can intervene remotely.

They have various means of intervention, that they can use according to the problem detected. In particular, they can trigger an alert on the worksite and they can also take control of a piece of equipment of the hydrocleaner, and in particular shut it down.

They also have one or more intervention vehicles 43 (FIG. 6) allowing a technician or a doctor to go to the worksite, if it is not too far away. The intervention vehicle 43 is in particular provided for proximity medical assistance.

The system 30, such as described hereinabove, makes it possible to monitor, from the control station 29, simultaneously a plurality of hydrocleaners working on the same site or on different sites that are far from one another.

The system 30 makes it possible to monitor any type of work carried out by the operator 3 with the hydrocleaner 2.

For the purposes of illustration, the hydrocleaner 2 such as shown in FIG. 2, comprises a pumping system 46, a water tank 47 for carrying out cleaning operations, and a collection tank 48 for collecting products pumped by the pumping system 46. II also comprises a suction hose 49 (for the pumping) and a high-pressure hose 50 (for cleaning). The hydrocleaner 2 thus comprises a set of operations equipment that makes it possible to perform pumping and cleaning operations. The hydrocleaner 20 further comprises the radio transmission units 5 and 8, alert elements, and sensors of sets 20 and 21, of which only one CC1 is shown in FIG. 2.

In the example of FIG. 2, the operator 3 is sucking products, for example oil products, via the suction hose 49. These products are sucked into the collection tank 48 of the hydrocleaner 2.

FIG. 5 shows another example of the work of the operator 3, with the hydrocleaner 3 of FIG. 2. In this example, the operator 3 is performing cleaning work using a high-pressure gun 51 supplied by the high-pressure hose 49 of the hydrocleaner 2.

For the purposes of illustration, non-limiting, mentioned is made hereinafter of different particular situations that can occur, and the monitoring, actions and/or interventions that can be performed in these particular situations.

Firstly, in case of a fall of the operator 3 on the worksite, as the operator 3 is constantly monitored thanks to the sensors incorporated into the piece or pieces of clothing 12 that they are wearing, the control station 29 is informed of this falling, via the low-frequency connection between the equipment of the operator 3 and the hydrocleaner 2 which is, itself, in liaison with the remotely-located control station 29.

Secondly, to respond to a risk of a dangerous atmosphere, at least one sensor worn by the operator 3 and at least one sensor installed on the hydrocleaner 2, inform the control station 29, which allows, instantly, if necessary, for taking control either medically or with regards to the equipment.

Thirdly, in case of abnormal operation at the hydrocleaner 2 (abnormal rise in temperature of the motor of the hydrocleaner 2 or of a pumping system 46, loss of oil pressure of the motor of the hydrocleaner 2 or of the drive of the pumping system 46, abnormal speed of the motor of the hydrocleaner 2 or of the pumping system 46, failure of the filling level of the collection tank 48, loss of equipotentiality of the suction line), the technician 40 of the control station 29 is informed, and they carry out the suitable actions such remotely taking control, and in particular shutting down the defective equipment.

Fourthly, in case of the bursting of a hose or a tool, the control station 29 is informed thanks to the monitoring of the entire line. If a sudden rupture in pressure is detected while the pump is delivering, the latter is automatically stopped. The state (medical) of the operator is also monitored by the control station 29.

Fifthly, when an operator is injured by the works, the constant monitoring of the operator 3, in particular at the heart level, immediately reports the accident, and the installation is shut down. At the same time, the doctor 41 in the control station 29 takes over the medical situation of the operator 3.

Consequently, in the control station 29, at each alert, a technician 40 and a doctor 41 provide the suitable technical support, even take control of the equipment, and the correct medical response with, if necessary, sending a team of rescue workers to the worksite.

1. An on-site monitoring device comprising a hydrocleaner, at least one monitoring unit for an operator of the hydrocleaner and a local monitoring unit mounted on the hydrocleaner, the hydrocleaner being intended for industrial installation and equipment cleaning work, the monitoring unit comprising:
at least one piece of clothing intended to be worn by the operator, said clothing being provided with at least one first set of sensors configured to measure values of parameters linked to said operator and/or to their environment; and
at least one radio transmitter configured to emit in the form of radio waves first information, and at least values measured by at least some of said sensors of said first set of sensors,
said local monitoring unit comprising at least one radio receiver configured to be able to receive the first information emitted in the form of radio waves by the radio transmitter of the monitoring unit and a radio transmission unit intended for communication with a remote control station, via a secure transmission.

2. The device according to claim 1, wherein said clothing corresponds to one of the following elements: a worksuit, an undergarment.

3. The device according to claim 1, wherein said first set of sensors comprises sensors able to measure at least some of the following parameters of the operator:
one or more pieces of heart data;
one or more pieces of respiratory data;
their body temperature;
their pulse;
their position.

4. The device according to claim 1, wherein said first set of sensors comprises sensors capable of measuring at least some of the following parameters of the environment of the operator:
the presence of at least one gas;
the temperature;
the wind.

5. The device according to claim 1, wherein said local monitoring unit comprises at least one second set of sensors configured to be mounted on the hydrocleaner, said second set of sensors comprising sensors configured to measure values of parameters linked to said hydrocleaner and/or to the environment thereof.

6. The device according to claim 5, wherein said second set of sensors comprises sensors capable of measuring at least some of the following parameters of the hydrocleaner:
parameters of a motor of the hydrocleaner;
parameters of a piece of equipment of the hydrocleaner.

7. The device according to claim 5, wherein said second set of sensors comprises sensors capable of measuring at least some of the following parameters of the environment of the hydrocleaner:
the presence of at least one gas;
the temperature;
the wind.

8. The device according to claim 1, wherein it comprises at least one alert element.

9. The device according to claim 1, wherein it comprises a central unit for processing the values measured.

10. A global monitoring system, wherein the system comprises at least one on-site monitoring device according to claim 1, and a remote control unit comprising at least one second radio transmission unit able to at least receive second information emitted by a first radio transmission unit of said local monitoring unit.

11. The system according to claim 10, wherein the remote control unit comprises at least one of the following elements:
at least one alert element;
at least one display element;
at least one data processing element.

12. The system according to claim 10, wherein the remote control unit comprises a control unit configured to generate control orders making it possible to remotely control at least one piece of equipment of the hydrocleaner, said control orders being emitted by said second radio transmission unit and received by said first radio transmission unit.

13. The system according to claim 10, wherein the remote control unit comprises an alert unit configured to generate orders for triggering an alert making it possible to remotely control at least one alert element of the on-site monitoring device, said orders for triggering an alert being emitted by said second radio transmission unit and received by said first radio transmission unit.
